(19) United States
(12) Patent Application Publication
(10) Pub. No.: US 2011/0152514 A1
(43) Pub. Date: Jun. 23, 2011

(54) DECOMPOSITION OF MATERIALS CONTAINING CARBOHYDRATES USING INORGANIC CATALYSTS

(75) Inventors: Ulrich Kettling, Munchen (DE); Andre Koltermann, Icking (DE); Michael Kraus, Puchheim (DE)

(73) Assignee: SUD-CHEMIE AG, Munich (DE)

(21) Appl. No.: 13/001,860

(22) PCT Filed: Jun. 30, 2009
(86) PCT No.: PCT/EP09/58144

§ 371 Date: Feb. 22, 2011

(30) Foreign Application Priority Data
Jun. 30, 2008 (DE) 102008030892.7

(51) Int. Cl. C07H 1/00

(52) U.S. Cl. 536/124

(57) ABSTRACT

The invention relates to a method for depolymerizing materials containing carbohydrates comprising the following steps: (a) treating a material containing carbohydrates with an inorganic catalyst in order to release defined monomeric or oligomeric building blocks from the material containing the carbohydrates; and (b) separating the defined monomeric or oligomeric building blocks produced in step (a) from the rest of the carbohydrate-containing material. Preferably, the inorganic catalyst used in step (a) comprises tectosilicates, phyilosilicates or hydrotalcites and more preferably zeolites or bentonites. The carbohydrate-containing material further comprises preferably LCB and the defined monomeric or oligomeric building blocks are preferably glucoses, xyloses, arabinoses and oligomers thereof. Other aspects of the invention refer to the use of solution promoters in combination with the inorganic catalyst.

### BACKGROUND OF THE INVENTION

The production of bio-based chemical building blocks from renewable sources is becoming increasingly important as a result of the global shortage of fossil petrochemical raw materials. The preferred starting materials for producing biologically based chemical products originate from renewable vegetable biomass.

The naturally occurring carbohydrate-containing materials are the most important representatives in the group of biological polymers which are typically referred to as “biomass”. Annual worldwide production by means of photosynthesis of plants is estimated at 1.3×10⁹ tons.

Current production methods for bio-based products are based primarily on substrates from the food and animal feed industry such as, for example, oils, sugars and starches. The majority of first-generation raw materials have a well-defined chemical composition and a low structural complexity. These substrates can additionally be obtained in a relatively high purity with only small quantities of accompanying contaminants. Although their use is both technically and commercially attractive, their ongoing availability on a large scale is not ensured since the use of first-generation raw materials for bio-based chemical methods is in stiff competition with the constantly increasing global demands of the food industry.

Alternative substrates to the above-mentioned first-generation raw materials are residues from the forestry and agriculture sectors such as, for example, wood and wood-related waste products, maize straw and wheat straw, herbaceous crops as well as solid municipal waste. These primarily comprise cellulose, hemicellulose and lignin and are referred to as lignocellulosic biomass (LCB). These alternative substrates involve vegetable materials which cannot be used as foodstuffs.

LCB differs from the biological first-generation raw materials in its complex chemical and structural composition. The main components of LCB are high polymer substances such as cellulose (approx. 35-50% by weight), hemicellulose (approx. 20-35% by weight) and lignins (approx. 10-25% by weight). Cellulose relates to a polysaccharide composed of β-1,4-glycosidically linked glucose monomers which is above all widespread in the plant world and usually occurs with other framework substances. Native cellulose comprises approx. 8,000 to 12,000 glucose units, corresponding to a relative molecular mass of 1.3 to 2.0 million. Purified cellulose is a colourless substance which is insoluble in water and the majority of organic solvents. In nature, cellulose never occurs as an individual chain, rather always as a crystalline conglomeration of a large number of chain microfibrils which are oriented in parallel.

In order to produce valuable chemical substances and building blocks on the basis of carbohydrate-containing material, it is important (i) to produce them with sufficient purity and (ii) with cost-effective methods.

Commercially valuable products can generally be produced from carbohydrate-containing materials. The depolymerisation products can serve both as raw materials for the production of further products, e.g. in chemical synthesis processes, and in the case of biotechnological conversions and are used in the chemical, cosmetic or pharmaceutical industry and the food industry.

In particular, the glucose obtained by depolymerisation from carbohydrate-containing materials, such as, for example, cellulose, is a versatile starting material for the production of high-quality chemical intermediate products such as, for example, sorbitol, lactate and ethanol. Pentose sugars obtained from hemicellulose fractions such as xylose and arabinose serve as the starting material for high-quality sugar alcohols such as xylitol and arabitol.

The natural structure of the cellulose brings about a high resistance of the cellulose to a depolymerisation. This applies both to chemical and enzymatic as well as microbial depolymerisation.

Enzymes or acid are used in the majority of common methods in which carbohydrate-containing materials are depolymerised.

The oldest methods for converting cellulose into glucose are based on an acid hydrolysis (Grethlein (1978), Chemical Breakdown of Cellulosic Materials, J. Appl. Chem. Biotechnol. 28: 296-308). In this method, concentrated acids are used at room temperature in order to dissolve the cellulose. A dilution to 1% strength acid and a one- to three-hour heating to 100° C. to 120° C. are subsequently carried out in order to convert cellulose oligomers into glucose monomers. This method produces a high yield of glucose, but recovery of the acid is a commercial problem of this method. Similar problems arise in the case of the use of organic solvents for the cellulose conversion.

U.S. Pat. No. 5,221,357-A and U.S. Pat. No. 5,536,325-A describe a two-stage method for the acid hydrolysis of lignocellulose-containing material to yield glucose in which diluted acids are used at high temperatures. Therein, in a first stage, hemicellulose is depolymerised to yield xylose and other sugars and in the second stage cellulose is depolymerised to yield glucose. The low acid content reduces the commercial necessity of a recovery of chemicals, nevertheless the maximum glucose content which can be achieved is low. Only up to 55% of the used cellulose is described in the literature.

Cellulose conversion methods have furthermore been developed which include a mechanical and/or chemical preliminary treatment and an enzymatic hydrolysis. The purpose of the preliminary treatment lies in destroying the fibre structures and improving the accessibility of the starting material for the cellulose enzymes used in the hydrolysis step. The mechanical preliminary treatment typically includes the use of pressure, grinding, milling, stirring, shredding, compression/expansion or other types of mechanical action. The chemical preliminary treatment typically includes the use of heat, often steam, acid, lyes and solvents. This combination of preliminary treatment with enzymatic hydrolysis involves high costs and has hitherto not been commercially competitive.

In JP 2006-129735, cellulose-containing materials with the addition of a carbon catalyst at temperatures of typically above 100° C. and lower than 300° C. are converted into glucose using catalytic depolymerisation methods.

The technical object on which the invention is based is consequently to develop a commercially attractive and environmentally friendly method with the help of which carbohydrate-containing materials are depolymerised in high concentrations and in mild reaction conditions into shorter chains as well as monomeric and oligomeric carbohydrates.

In particular, the technical problem lies in providing a method for producing valuable chemical building blocks from LCB which avoids the disadvantages and drawbacks of the prior art.

### SUMMARY OF THE INVENTION

The invention relates to a method for producing basic chemicals or chemical building blocks composed of polymeric or oligomeric carbohydrate-containing material such as, for example, LCB. In the method, soluble monomeric or oligomeric building blocks are released from carbohydrate-containing material in that the carbohydrate-containing material is brought into contact with an inorganic catalyst, preferably a tectosilicate, phyllosilicate or hydrotalcite, in a solvent system.

The problems which arise from the prior art are solved according to the invention in that carbohydrate-containing materials are brought into contact with suitable inorganic catalysts and the conversion into shorter chains and monomeric and oligomeric carbohydrates is carried out in suitable solvent systems and in mild ambient conditions.

According to a first aspect, the present invention supplies a method for catalytic treatment of a carbohydrate-containing material, comprising the following steps: (a) treating the carbohydrate-containing material with an inorganic catalyst, (b) releasing defined monomeric or oligomeric building blocks from the polymeric carbohydrate-containing material by means of the catalyst; and (c) separating the defined monomeric or oligomeric building blocks produced in step (b) from the residue of the carbohydrate-containing material.

One advantage of the present invention is the fact that there is no need for expensive enzymes for the depolymerisation of carbohydrate-containing materials.

Further preferred aspects and embodiments are described in detail below.

### DETAILED DESCRIPTION OF THE INVENTION

In the first step of the method according to the invention, a carbohydrate-containing material is treated with an inorganic catalyst in order to release defined monomeric or oligomeric building blocks from the carbohydrate-containing material.

The term “carbohydrate-containing material” includes pure substances containing carbohydrate, mixtures of various carbohydrates as well as complex mixtures of substrates which contain carbohydrates. Carbohydrate-containing material furthermore includes, but is not restricted to, waste products from forestry and agriculture and the food-processing industries as well as municipal waste. In particular, “lignocellulosic biomass” or “LCBs” fall under the carbohydrate-containing materials. This includes carbohydrate-containing material which contains cellulose, hemicellulose and lignin. The insoluble fraction of the LCB generally contains significant quantities of polymeric substrates such as cellulose, xylan, mannan and galactan. It additionally contains polymeric substrates such as lignin, arabinoxylan, glucoronoxylan, glucomannan and xyloglucan.

LCBs from agriculture include, but are not restricted to, wheat straw, maize straw, manure from ruminants, sugar press cake, sugar beet pulp and herbaceous materials such as barley grass, Sericea Lespedeua Serala and Sudan grass. LCBs in the form of waste products from forestry include, but are not restricted to, tree bark, wood chip and wood cuttings. LCBs in the form of raw substrates from the food industry include, but are not restricted to, fruit pulp, agave residues, coffee residues and oil mill waste such as rape seed press cakes and mill waste water. LCBs in the form of raw substrates from the pulp and paper industry include, but are not restricted to, pulp and paper mill waste water. LCBs in the form of raw substrates from municipal waste include, but are not restricted to, paper waste, vegetable residues and fruit residues.

According to a preferred embodiment of the invention, the carbohydrate-containing material involves material containing cellulose and/or hemicellulose, in particular one or more LCBs.

The carbohydrate-containing material can be milled prior to the treatment according to the invention with the catalyst.

The inorganic catalyst is preferably a silicate or clay material which is preferably doped with impurity ions.

The term “tectosilicate”, as used in the present invention, includes any tectosilicate known to a person skilled in the art and in particular any zeolite. Possible structures and examples of numerous tectosilicates and in particular zeolites are explained, for example, in “Holleman-Wiberg, Lehrbuch der Anorganischen Chemie” by N. Wiberg, 91^{st} to 100^{th} Edition, Walter de Gruyter & Co., 1985, ISBN 3-11-007511-3, pp. 776 to 778. Zeolites and their representation are furthermore explained in “Römpp-Lexikon Chemie”, Ed.: J. Falbe, M. Regitz, 10^{th} Edition 1999, Georg Thieme Verlag, ISBN 3-13-107830-8, p. 5053 ff.

In particular, the term “tectosilicate” includes all compounds in which silicon atoms are replaced partially by other atoms, in particular aluminium, in the web structure of the silicon dioxide. Preferably at least 1%, preferably at least 5%, more preferably at least 8%, more preferably at least 12% of the silicon atoms of the tectosilicate can be replaced by aluminium atoms. Furthermore, a tectosilicate, in particular a zeolite, can have cavities and/or channels which connect the cavities at least partially to one another, wherein the cavities can have, for example, a diameter of 350 to 1300 pm and the channels can have, for example, a diameter of 180 to 800 pm. In particular, the one or more tectosilicates can involve one or more zeolites or mixtures of zeolite(s) with further tectosilicates.

In particular, the inorganic catalyst can include one or more zeolites, for example, in addition to optional other tectosilicates or can be composed of these. According to a preferred embodiment, the inorganic catalyst includes one or more zeolites which are selected from the group comprising fibrous zeolites, leaf zeolites, cubic zeolites, zeolites of MFI structure type, zeolite A, zeolite X, zeolite Y and mixtures thereof. Fibrous zeolites include, for example, among other things, natrolite, laumontite, mordenite, thomsonite, leaf zeolites include, among other things, heulandite, stilbite and cubic zeolites include, among other things, faujasite, chabazite and gmelinite.

Possibilities for obtaining naturally occurring zeolites as well as methods for producing synthetic zeolites are known to a person skilled in the art. Methods for producing synthetic zeolites with an MFI structure, with a Si/Al atomic ratio of approximately 8 to 45 are, for example, described in WO 01/30697.

The term “phyllosilicate”, as used in the present invention, includes any phyllosilicate known to a person skilled in the art and in particular any smectitic silicate. For example, reference can be made to “Römpp-Lexikon Chemie”, Ed.: J. Falbe, M. Regitz, 10^{th} Edition 1998/1999, Georg Thieme Verlag, ISBN 3-13-107830-8, p. 3328/3329 and p. 4128.

Particularly preferred phyllosilicates are bentontites whose main mineral is montmorillonite and other montmorillonite-containing phyllosilicates as well as other smectitic clay minerals such as beidellite, saponite, glauconite, nontronite and hectorite. The phyllosilicates or bentonites used according to the invention preferably contain 70 to 80% by weight montmorillonite. Particularly preferred bentonites are acid-activated bentonites. Likewise particularly preferred bentonites are alkali-activated bentonites.

Bentonites exhibit surprisingly improved properties in comparison to the known carbon catalysts in terms of the concentration and temperature ranges required for a catalytic depolymerisation. The quantities of the catalyst required for a depolymerisation in the methods described according to the invention are also significantly smaller than for the known carbon catalysts.

The term “hydrotalcite” is familiar to the person skilled in the art and refers to synthetically produced aluminium/magnesium hydroxycarbonates.

For the purposes of the invention, it is advantageous according to a preferred embodiment if inorganic catalysts contain in addition to Al further elements of the 3^{rd} main group such as e.g. Ga, B or In. H⁺, Na⁺, Li⁺, K⁺, Rb⁺, Cs⁺, NH₄⁺, Mg²⁺, Ca²⁺, Sr²⁺ and Ba⁺ can be contained in the catalyst as counterions for the excess negative charge produced by the trivalent framework cations. The catalysts can furthermore in addition to Si contain further elements of the 4^{th} main or subsidiary group such as Ti, Ge or Sn.

According to a preferred embodiment according to the invention, the inorganic catalysts are doped with impurity ions or impurity atoms prior to the use of methods known to the person skilled in the art. The impurity ions or impurity atoms can be applied by wet chemical means in the form of aqueous, organic or organic-aqueous solutions of their salts by impregnation of the catalysts with the saline solution. The wet chemical treatments are typically followed by drying in a vacuum at approximately 100° C. and thereupon calcination at approximately 400 to 800, preferably, however, below 600° C., for example, for 0.1 to 24 hours. The impurity ions can furthermore also be applied onto the catalysts by dry chemical means, for example, in that a compound which is gaseous at higher temperatures is separated out from the gas phase on the catalyst. Nickel, cobalt, platinum, palladium, gallium or indium are preferably used as impurity ions. Platinum has proved to be particularly suitable in particular for zeolite catalysts and gallium for bentonite catalysts.

The doping with impurity ions is preferably carried out in a quantity of 0.1 to 10% by weight, particularly preferably 0.2 to 5% by weight relative to the weight of the silicate or clay material.

In the context of the present invention, active carbon is not regarded as an inorganic catalyst. According to a preferred embodiment, inorganic catalysts furthermore exclude catalysts with at least one C—H bond.

The catalyst is preferably present in particulate form, particularly preferably in a particle size of 1 to 100 μm.

In the method according to the invention, the catalyst is preferably used in a quantity of 1 to 20% by weight, preferably 2 to 15% by weight, particularly preferably 6 to 12% by weight relative to the carbohydrate-containing material.

For the purposes of the invention, it is advantageous if the depolymerisation is carried out at low temperatures and pressures. The temperatures preferably lie between 20° C. and 400° C., particularly preferably between 20° C. and 150° C., particularly preferably between 100° C. and 140° C. The pressure preferably lies between 0 bar and 200 bar, particularly preferably between 0 bar and 5 bar.

According to a further preferred embodiment, the carbohydrate-containing material is present in a solvent system. The solvent system preferably comprises one or more organic or inorganic solvents. Therein, water or alcohols with 2 to 6 carbon atoms are particularly preferred.

According to a further preferred embodiment according to the invention, the solvent system involves an aqueous system which preferably contains solubilizers such as, for example, detergents. According to a further preferred embodiment, the solvent system furthermore contains at least one acid, in particular a strong inorganic acid, more preferably hydrochloric acid (HCl) or sulphuric acid. The quantity of acid in the solvent system preferably lies between approximately 0.1 and 5% by weight, more preferably between approximately 0.5 and 2% by weight relative to the total quantity of solvent system. The solvent should preferably be added in a quantity of 1 to 10 litres, preferably 2 to 5 litres per 1 kg carbohydrate-containing material.

Instead of a single inorganic catalyst, a mixture of two or more inorganic catalysts and solvent systems can also advantageously be used.

The term “solubilizer” includes all detergents which increase the solubility characteristics of cellulose-containing materials in liquid solvent systems. In particular, this includes non-ionic, anionic, cationic and amphoteric detergents. Particularly suitable anionic detergents include alkyl (ether) sulphates such as, for example, lauryl sulphate or lauryl ether sulphate. Non-ionic detergents include in particular polyethylene ethers or polypropylene ethers such as e.g. Tween 20 or Triton-X 100 as well as triethanol amine. The detergents are preferably used in a quantity of 0.1 to 0.5% by weight relative to the solvent.

According to the invention, monomeric or oligomeric building blocks are released from the carbohydrate-containing material. The term “monomeric or oligomeric building blocks” refers to monomeric or oligomeric products which are released from the carbohydrate-containing material using an inorganic catalyst. The term “oligomer” includes compounds with at least two and/or up to 20 monomeric units. The term “release” or “depolymerise” refers to the conversion of a polymeric substrate into soluble monomeric or oligomeric building blocks by means of a physical, chemical or catalytic method such as, for example, hydrolysis, oxidative or reductive depolymerisation as well as further methods known to the person skilled in the art.

According to one preferred embodiment, the defined monomeric or oligomeric building block(s) which are released from the carbohydrate-containing raw substrate in step (b) is/are glucose, xylose, arabinose and/or oligomers which are constructed from monomeric glucose building blocks.

After treatment with the catalyst, the monomeric and/or oligomeric building blocks are separated from the rest of the carbohydrate-containing material. When using e.g. water as the solvent, these building blocks are soluble in the solvent so that separation by fluid/solid separation of the soluble building stones can be carried out in the aqueous medium from the insoluble carbohydrate-containing raw substrate.

Methods for separating soluble and insoluble components are known to the person skilled in the art and include method steps such as sedimentation, decantation, filtration, microfiltration, ultrafiltration, centrifugation, evaporation of volatile products and extraction with organic solvents. According to one preferred embodiment, the physical-chemical treatment step includes a treatment with aqueous solvents, organic solvents or any combination or any mixture of these, preferably with ethanol or glycerine.

A further aspect of the present invention relates to the use of an inorganic catalyst, in particular selected from the group comprising tectosilicates, phyllosilicates, hydrotalcites and mixtures thereof for the treatment, in particular the depolymerisation of a carbohydrate-containing material.

The invention is explained in greater detail below with reference to non-restrictive examples.

### EXAMPLE 1

1 g cellulose (Avicel PH-101; Fluka, Buchs) is suspended with 100 mg of the zeolite Wessalith DAY P (Degussa/Evonic, Essen) as the inorganic catalyst and 2 ml distilled H₂O with or without the addition of 1% HCl in a pressure vessel (5 ml) and stirred for 1 min. at 20° C. This mixture is then heated for 20 min to 120° C. After cooling of the mixture to room temperature, the solid and the liquid phase are separated by centrifugation. The cellulose content in the solid phase is determined gravimetrically after drying and the glucose content in the liquid phase is determined by HPLC (Aminex HPX-87C; Bio-Rad, Munich). The yield of glucose is increased by up to 35% on a molar basis in comparison to an approach without addition of the catalyst.

### EXAMPLE 2

10 g cellulose (Avicel PH-101; Fluka, Buchs) is suspended with 1 g of a bentonite dealuminised with acid (Tonsil Supreme 110F, Süd-Chemie, Munich) and 20 ml distilled H₂O with or without the addition of 1% HCl in a pressure vessel and stirred for 1 min. at 20° C. This mixture is then heated for 20 min to 135° C. After cooling of the mixture to room temperature, the solid and the liquid phase are separated by centrifugation. The cellulose content in the solid phase is determined gravimetrically after drying and the glucose content is determined by HPLC (Aminex HPX-87C; Bio-Rad, Munich). The yield of glucose in the liquid phase is increased by up to 27% on a molar basis in comparison to an approach without addition of the catalyst.

### EXAMPLE 3

1 g of the zeolite Wessalith DAY P (Degussa/Evonic, Essen) is intensively mixed with 100 mg PTCl₂ (Sigma Aldrich, Munich) in a vibromill over a period of 2 h. The mixture is subsequently calcinated at a temperature of 550° C. The heating temperature is 10 K/min. 1 g cellulose (Avicel PH-101; Fluka, Buchs) is suspended with 100 mg of the zeolite, which is doped as described above, and 2 ml distilled H₂O with or without the addition of 1% HCl in a pressure vessel (5 ml) and stirred for 1 min. at 20° C. This mixture is then heated for 20 min to 100° C. After cooling of the mixture to room temperature, the solid and the liquid phase are separated by centrifugation. The cellulose content in the solid phase is determined gravimetrically after drying and the glucose content is determined by HPLC (Aminex HPX-87C; Bio-Rad, Munich). The yield of glucose in the liquid phase is increased by up to 56% on a molar basis in comparison to an approach without addition of the catalyst.

### EXAMPLE 4

1 g of a bentonite dealuminised with acid (Tonsil Supreme 110F, Süd-Chemie, Munich) is sprayed with 20 μl of a 25% strength gallium sulphate solution. The impregnated bentonite is dried for 24 hours at 120° C. 1 g cellulose (Avicel PH-101; Fluka, Buchs) is suspended with 100 mg of the bentonite, which is Ga-substituted as described above, and 2 ml distilled H₂O with or without the addition of 1% HCl in a pressure vessel (5 ml) and stirred for 1 min at 20° C. This mixture is then heated for 20 min to 110° C. After cooling of the mixture to room temperature, the solid and the liquid phase are separated by centrifugation. The cellulose content in the solid phase is determined gravimetrically after drying and the glucose content is determined by HPLC (Aminex HPX-87C; Bio-Rad, Munich). The yield of glucose in the liquid phase is increased by up to 75% on a molar basis in comparison to an approach without addition of the catalyst.

### EXAMPLE 5

100 mg of the bentonite from Example 4 is suspended with 1 g cellulose (Avicel PH-101; Fluka, Buchs) and 2 ml distilled H₂O, which contains a detergent (0.25% Triton-X 100), in a pressure vessel (5 ml) and stirred for 1 min at 20° C. This mixture is then heated for 20 min to 110° C. After cooling of the mixture to room temperature, the solid and the liquid phase are separated by centrifugation. The cellulose content in the solid phase is determined gravimetrically after drying and the glucose content is determined by HPLC (Aminex HPX-87C; Bio-Rad, Munich). The yield of glucose in the liquid phase is increased by up to 50% on a molar basis in comparison to an approach without addition of the detergent.

### EXAMPLE 6

10 g cellulose (Avicel PH-101; Fluka, Buchs) is suspended with 1 g of an acidic bentonite (Tonsil Supreme 110F, Süd-Chemie, Munich) and 20 ml distilled H₂O with 1% (w/w) H₂SO₄ in a pressure vessel and stirred for 1 min at 20° C. This mixture is then heated for 20 min to 135° C. After cooling of the mixture to room temperature, the solid and the liquid phase are separated by centrifugation. The cellulose content in the solid phase is determined gravimetrically after drying and the glucose content is determined by HPLC (Aminex HPX-87C; Bio-Rad, Munich). The yield of glucose in the liquid phase is increased by up to 22% on a molar basis in comparison to an approach without addition of the catalyst.

1. Method for treating a carbohydrate-containing material with an inorganic catalyst, comprising the following steps:
a. treating the carbohydrate-containing material with an inorganic catalyst in order to release defined monomeric or oligomeric building blocks from the carbohydrate-containing material;
b. separating the defined monomeric or oligomeric building blocks produced in step a. from the rest of the carbohydrate-containing material.

2. Method according to claim 1, wherein tectosilicates, phyllosilicates or hydrotalcite are used as the catalyst.

3. Method according to one of the preceding claims, wherein zeolites or bentonites are used as the catalyst.

4. Method according to one of the preceding claims, wherein doped inorganic catalysts are used.

5. Method according to one of the preceding claims, wherein the carbohydrate-containing material is preliminarily treated in at least one physical, chemical or physicochemical step.

6. Method according to one of the preceding claims, wherein the treatment of the carbohydrate-containing material is carried out with an inorganic catalyst in a solvent system.

7. Method according to claim 6, wherein the solvent system contains at least one solubilizer.

8. Method according to one of the preceding claims, wherein the solvent system contains at least one acid, in particular a strong inorganic acid, more preferably hydrochloric acid.

9. Method according to claim 8, wherein the quantity of acid in the solvent system lies between approximately 0.1 and 5% by weight, more preferably between approximately 0.5 and 2% by weight relative to the total quantity of solvent system.

10. Use of an inorganic catalyst, selected from the group comprising tectosilicates, phyllosilicates, hydrotalcites and mixtures thereof for the treatment, in particular the depolymerisation of a carbohydrate-containing material.
